SEQUENCE LISTING (22 sequences)

SEQ ID NO: 1 - DNA, 2713 bp, Artificial
  multi-transgene DNA sequence ISC
ggaagaaggcgaagtcatgagcgccgggatttaccccctaacctttatataagaaacaatggatattactgctacagggacccaaggacgggtaaagagtttggattaggcagagacaggcgattcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacgctgagcgatgcattccgagaggcaatagctgaaggccatataacaacaaaccatgtcgctgccactcgcgcggcaaagtcaaaggtaaggagatcaagacttacggctgacgaatacctgaaaatttatcaagcagcagaatcatcaccatgttggctcagacttgcaatggaactggctgttgttaccgggcaacgagttggtgacttgtgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctctcatccggcacagtatcaaggtattttatgcgcgcacgaaaagcatcaggtctttccttcgaaggggatccgcctacctttcacgagttgcgcagtttgtctgcaagactctatgggaagcagataagcgataagtttgctcaacatcttctcgggcataagtcggtcaccatggcatcacagtatcgtgatgacagaggcagggagtgggacaaaattgaaatcaaatgaaaaacgaatggctagcaccaagtcagaattgatagaaagacttgccacccagcaatcgcacattcccgccaagacggttgaagatgcagtaaaagagatgctggagcatatggcctcgactcttgcgcagggtggaagcggcggtcttacaaaagctgaaatgtcagaatatctgtttgataagcttgggcttagcaagcgggatgccaaagaactggttgaactgtttttcgaagagatccgtcgcgctctggaaaacggcgaacaggtgaaactctctggttttggtaacttcgatctgcgtgataagaatcaacgcccgggacgtaacccgaaaacgggcgaggatattcccattacagcacggcgcgtggtgaccttcagacccgggcagaagttaaaaagccgggtcgaaaacgctggtgggggcgagcgtattgaaatccgcggtttcggcagtttctctttgcactaccgcgcaccacgtaccggacgtaatccgaagactggcgataaagtagaactggaaggaaaatacgttcctcactttaaacctggtaaagaactgcgcgatcgcgccaatatctacggtggatcaggctgataaataaaacgaaaggctcagtcgaaagactgggcctttcgttttatcctattccgaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggcgcgccatttaaatgaagttcctattccgaagttcctattctctagaaagtataggaacttcgactgtataaaaccacagcc

SEQ ID NO: 2 - protein, 356 aa, Artificial
  mutant INTC3 recombinase
MGRRRSHERRDLPPNLYIRNNGYYCYRDPRTGKEFGLGRDRRFAITEAIQANIELFSGHKHKPLTARINSDNSVTLHSWLDRYEKILASRGIKQKTLINYMSKIKAIRRGLPDAPLEDITTKEIAAMLNGYIDEGKAASAKLIRSTLSDAFREAIAEGHITTNHVAATRAAKSKVRRSRLTADEYLKIYQAAESSPCWLRLAMELAVVTGQRVGDLCKMKWSDIVDGYLYVEQSKTGVKIAIPTALHIDALGISMKETLDKCKEILGGETIIASTRREPLSSGTVSRYFMRARKASGLSFEGDPPTFHELRSLSARLYGKQISDKFAQHLLGHKSVTMASQYRDDRGREWDKIEIK

SEQ ID NO: 3 - DNA, 72 bp, Artificial
  Primer sequence
actctctactgtttctccatacccgtttttttggatggagtgaaacgatgggaagaaggcgaagtcatgagc

SEQ ID NO: 4 - DNA, 72 bp, Artificial
  Primer sequence
gccaaagctcgcacagaatcactgccaaaatcgaggccaattgcaatcgcttatacagtcgaagttcctata

SEQ ID NO: 5 - DNA, 25 bp, Artificial
  PCR primer
gtctataatcacggcagaaaagtcc

SEQ ID NO: 6 - DNA, 21 bp, Artificial
  PCR primer
tcgcctgtctctgcctaatcc

SEQ ID NO: 7 - DNA, 19 bp, Artificial
  PCR primer
cgcaaggcgacaaggtgct

SEQ ID NO: 8 - DNA, 25 bp, Artificial
  PCR primer
ccgcttccattgactcaatgtagtc

SEQ ID NO: 9 - protein, 198 aa, Artificial
  scIHF2
MASTKSELIERLATQQSHIPAKTVEDAVKEMLEHMASTLAQGGSGGLTKAEMSEYLFDKLGLSKRDAKELVELFFEEIRRALENGEQVKLSGFGNFDLRDKNQRPGRNPKTGEDIPITARRVVTFRPGQKLKSRVENAGGGERIEIRGFGSFSLHYRAPRTGRNPKTGDKVELEGKYVPHFKPGKELRDRANIYGGSG

SEQ ID NO: 10 - DNA, 1071 bp, Artificial
  IntC3 coding sequence
atgggaagaaggcgaagtcatgagcgccgggatttaccccctaacctttatataagaaacaatggatattactgctacagggacccaaggacgggtaaagagtttggattaggcagagacaggcgattcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacgctgagcgatgcattccgagaggcaatagctgaaggccatataacaacaaaccatgtcgctgccactcgcgcggcaaagtcaaaggtaaggagatcaagacttacggctgacgaatacctgaaaatttatcaagcagcagaatcatcaccatgttggctcagacttgcaatggaactggctgttgttaccgggcaacgagttggtgacttgtgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctctcatccggcacagtatcaaggtattttatgcgcgcacgaaaagcatcaggtctttccttcgaaggggatccgcctacctttcacgagttgcgcagtttgtctgcaagactctatgggaagcagataagcgataagtttgctcaacatcttctcgggcataagtcggtcaccatggcatcacagtatcgtgatgacagaggcagggagtgggacaaaattgaaatcaaataa

SEQ ID NO: 11 - DNA, 261 bp, Artificial
  attP recombination site
tctgttacaggtcactaataccatctaagtagttgattcatagtgactgcatatcttgtgttttacagtattaattagtctgttttttatccaaaatctaatttaatatattgatatttatatcattttacgtttctcgttcagcttttttatactaagttggcattataaaaaagcattgcttatcaatttgttgcaacgaacaggtcactatcagtcaaaatacaatcattatttgatttcaattttgtcccactccct

SEQ ID NO: 12 - DNA, 21 bp, Artificial
  attB recombination site
ctgcttttttatactaacttg

SEQ ID NO: 13 - DNA, 120 bp, Artificial
  attL recombination site
ctgcttttttatactaagttggcattataaaaaagcattgcttatcaatttgttgcaacgaacaggtcactatcagtcaaaatacaatcattatttgatttcaattttgtcccactccct

SEQ ID NO: 14 - DNA, 18 bp, Artificial
  attH4x recombination site
ctttatttcattaagttg

SEQ ID NO: 15 - DNA, 271 bp, Artificial
  attP4x recombination site
aattcctctgtaacaggtcactaataccatctaagtagttgattcatagtgactgcatatgttgtgttttacagtattatgtagtctgttttttatgcaaaatctaatttaatatattgatatttatatcattttacgtttctcgttcagctttatttcattaagttggcattataaaaaagcattgcttatcaatttgttgcaacgaacaggtcactatcagtcaaaatacaatcattatttgatttcaattttgtcccactccctcccg

SEQ ID NO: 16 - DNA, 121 bp, Artificial
  attL4x recombination site
ctttatttcattaagttggcattataaaaaagcattgcttatcaatttgttgcaacgaacaggtcactatcagtcaaaatacaatcattatttgatttcaattttgtcccactccctcccg

SEQ ID NO: 17 - DNA, 168 bp, Artificial
  attR4x recombination site
aattcctctgtaacaggtcactaataccatctaagtagttgattcatagtgactgcatatgttgtgttttacagtattatgtagtctgttttttatgcaaaatctaatttaatatattgatatttatatcattttacgtttctcgttcagctttatttcattaagttg

SEQ ID NO: 18 - DNA, 595 bp, Artificial
  scIHF2
atggctagcaccaagtcagaattgatagaaagacttgccacccagcaatcgcacattcccgccaagacggttgaagatgcagtaaaagagatgctggagcatatggcctcgactcttgcgcagggtggaagcggcggtcttacaaaagctgaaatgtcagaatatctgtttgataagcttgggcttagcaagcgggatgccaaagaactggttgaactgtttttcgaagagatccgtcgcgctctggaaaacggcgaacaggtgaaactctctggttttggtaacttcgatctgcgtgataagaatcaacgcccgggacgtaacccgaaaacgggcgaggatattcccattacagcacggcgcgtggtgaccttcagacccgggcagaagttaaaaagccgggtcgaaaacgctggtgggggcgagcgtattgaaatccgcggtttcggcagtttctctttgcactaccgcgcaccacgtaccggacgtaatccgaagactggcgataaagtagaactggaaggaaaatacgttcctcactttaaacctggtaaagaactgcgcgatcgcgccaatatctacggtggatcaggct

SEQ ID NO: 19 - DNA, 647 bp, Artificial
  chloramphenicol resistance gene
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggca

SEQ ID NO: 20 - DNA, 34 bp, Artificial
  FLP-FRT recombination site
gaagttcctattctctagaaagtataggaacttc

SEQ ID NO: 21 - DNA, 63 bp, Artificial
  partial genomic sequence
tcactgccaaaatcgaggccaattgcaatcgccatcgtttcactccatccaaaaaaacgggta

SEQ ID NO: 22 - DNA, 63 bp, Artificial
  partial genomic sequence
tacccgtttttttggatggagtgaaacgatggcgattgcaattggcctcgattttggcagtga

(19) United States
(12) Patent Application Publication
(10) Pub. No.: US 2024/0175038 A1
(43) Pub. Date: May 30, 2024

(54) METHOD FOR THE PRODUCTION OF SEAMLESS DNA VECTORS

(71) Applicant: Nanyang Technological University, Singapore (SG)

(72) Inventors: Peter Droge, Singapore (SG); Sabrina d/o Peter, Singapore (SG); Suki Roy, Singapore (SG)

(21) Appl. No.: 18/549,856

(22) PCT Filed: Mar. 11, 2022
(86) PCT No.: PCT/SG2022/050125

§ 371 Date: Sep. 8, 2023

(30) Foreign Application Priority Data
Mar. 12, 2021 (SG) 10202102572X

(51) Int. Cl. C12N 15/70; C12N 1/20; C12N 9/12; C12N 15/64; C12N 15/66; C12R 1/19

(52) U.S. Cl. CPC C12N 15/70; C12N 1/205; C12N 9/1241; C12N 15/64; C12N 15/66; C12N 2501/90; C12R 2001/19

(57) ABSTRACT

The present invention relates to a method for in vivo production of seamless DNA vectors in E. coli, said seamless DNA vectors comprising a DNA sequence of interest and a phage lambda integrase recombination sequence. The method comprises providing an E. coli strain encoding a mutant phage lambda integrase (IntC3), stringently controlled by an inducible expression control sequence; transforming into the E. coli strain a bacterial plasmid comprising the DNA sequence of interest and a bacterial backbone sequence flanked by two directly repeated lambda integrase recombination sequences, wherein the bacterial backbone sequence comprises a selection marker; cultivating the transformed E. coli cells under conditions selective for the selection marker; inducing the expression of IntC3 to facilitate recombination to obtain a dimeric DNA catenane consisting of a first circular DNA molecule carrying the bacterial backbone and a second circular DNA molecule carrying the DNA sequence of interest and the phage lambda integrase recombination sequence.

### CROSS-REFERENCE TO RELATED APPLICATION

This application claims the benefit of priority of Singapore Patent Application No. 10202102572X filed Mar. 12, 2021, the content of which being hereby incorporated by reference in its entirety for all purposes.

### FIELD OF THE INVENTION

The present invention relates generally to the field of DNA vector production by recombinant expression in E. coli host cells, and more specifically to the production of seamless DNA vectors by regulated expression of enhanced phage lambda integrase in E. coli, as well as the E. coli strains engineered and used in said methods.

### BACKGROUND OF THE INVENTION

Seamless DNA vectors—also frequently referred to as “minicircle vectors”—are derived from bacterial plasmids and devoid of bacterial genetic elements such as origins of replication and resistance markers which are important for episomal plasmid growth/maintenance. Seamless vectors are circular, covalently closed and negatively supercoiled DNA molecules and becoming increasingly attractive for various biomedical applications such as cell line engineering, biologics production, gene/cell therapy and DNA vaccination. This is due to their enhanced transgene expression, improved safety features, reduced gene silencing, and higher gene transfer efficiency when compared to the parental bacterial plasmids [Chen Z Y, Riu E, He C-Y et al (2008) Mol Ther 16:548-5561; Kay M A (2011) Nat Rev Genet 12:316-328].

The most common means to produce seamless DNA vectors are site-specific DNA recombinases which utilize their respective cognate DNA sequences that flank the unwanted bacterial genetic elements. Through a precise DNA strand cutting and pasting reaction, these enzymes splice out intervening DNA from the rest of the molecule. The recombination reaction using plasmids as substrate thus results in two circular DNA molecules: one that carries the unwanted bacterial elements and the other representing the desired seamless vector. Various protocols are available so that the latter can be isolated and purified for downstream purposes.

The site-specific recombination reaction can be carried out either in vitro with purified enzymes for small scale production or in vivo (e.g. inside the bacterium Escherichia coli) to achieve medium to large scale production, the latter currently being employed in commercial settings. A number of recombinases have been utilized for in vivo production of seamless vectors in E. coli and include, for example, the wild-type phage lambda integrase [Darquet A, Cameron B, Wils P et al. (1997) Gene Ther 4:1341-1349], the yeast recombinases Cre and FLP [Bigger B W, Tolmachov O, Collombet J M et al. (2001) J Biol Chem 276:23018-23027; Nehlsen K, Broll S, Bode J et al (2006) Gene Ther Mol Biol 10:233-244], the wild-type phage ϕC31 integrase [Chen Z Y, He C Y, Ehrhardt A, Kay M A et al (2003) Mol Ther 8:495-500], and the ParA resolvase [Jechlinger W, Azimpour Tabrizi C, Lubitz W et al (2004) J Mol Microb Biotech 8:222-231]. Wild-type lambda integrase and its in vivo use has also been described in U.S. Pat. No. 6,143,530A; WO1994/009127 A2; US20130316449A1. The use of a mutant lambda integrase, termed IntC3, for small scale in vitro production of seamless vectors has been described in US 2017/0362606 A1.

For in vivo seamless vector production, a major technical challenge is the stringent control of the expression of the recombinase. Leaky expression during bacterial growth will result in premature loss of the seamless vector inside the bacterium and hence severely compromise vector yield. In order to achieve this, several expression systems have been employed including temperature-sensitive lambda repressor cI857/pR [Darquet A, Cameron B, Wils P et al (1997) Gene Ther 4:1341-1349] and the plasmid-based pBAD/araC arabinose system [Chen Z Y, He C Y, Ehrhardt A, Kay M A et al (2003) Mol Ther 8:495-500]. However, employing these systems reproducibly and, in particular, at larger scales still remains technically challenging.

### SUMMARY OF THE INVENTION

The inventors have engineered a novel E. coli strain derived from strain MG1655 that can be used for multi-scale and multi-purpose seamless vector production by in vivo site-specific recombination catalyzed by mutant lambda integrase IntC3 (a mutant lambda integrase being described in WO 2016/022075 A1).

In one aspect, the present application therefore relates to a method for the in vivo production of seamless DNA vectors in E. coli, said seamless DNA vectors comprising a DNA sequence of interest and a phage lambda integrase recombination sequence, the method comprising:
(i) providing an E. coli strain comprising a nucleotide sequence encoding a mutant phage lambda integrase (IntC3) having the amino acid sequence set forth in SEQ ID NO:2 or a functional variant or fragment thereof, wherein the expression of said nucleotide sequence is stringently controlled by an inducible expression control sequence;
(ii) transforming a bacterial plasmid comprising a DNA sequence of interest and a bacterial backbone sequence flanked by two directly repeated lambda integrase recombination sequences that are recombination substrates for the mutant phage lambda integrase into the E. coli strain of (i), wherein the bacterial backbone sequence comprises a selection marker;
(iii) cultivating the transformed E. coli cells under conditions selective for the selection marker comprised in the bacterial plasmid;
(iv) inducing the expression of the mutant phage lambda integrase to facilitate recombination of the two directly repeated lambda integrase recombination sequences in the bacterial plasmid to obtain a dimeric DNA catenane consisting of a first circular DNA molecule that carries the bacterial backbone and a second circular DNA molecule that carries the DNA sequence of interest and a phage lambda integrase recombination sequence that is a hybrid of the two directly repeated lambda integrase recombination sequences; and
(v) isolating the second circular DNA molecule that carries the DNA sequence of interest and a phage lambda integrase recombination sequence.

In various embodiments, step (iv) comprises unlinking the two catenated circular DNA molecules.

Step (v) may comprise linearizing the first circular DNA molecule, i.e. the DNA molecule comprising the bacterial backbone of the plasmid. In such embodiments, the isolation in step (v) may comprise digesting the linearized DNA and optionally also any nicked circular DNA. Said step may also comprise extraction of the second circular DNA molecule and/or its purification/separation from any other unwanted cellular components.

In various embodiments, the nucleotide sequence encoding a mutant phage lambda integrase having the amino acid sequence set forth in SEQ ID NO:2 or a functional variant or fragment thereof is stably integrated into the E. coli strains genome.

The inducible expression control sequence may be the E. coli arabinose operon. In such embodiments, the induction in step (iv) may be triggered by the addition of arabinose. If the inducible expression control sequence is the E. coli arabinose operon, the nucleotide sequence encoding a mutant phage lambda integrase (IntC3) may be inserted into the genomic arabinose operon of E. coli. The insertion may occur immediately downstream of the arabinose promoter, for example by using the start codon of the endogenous araB gene as the start codon for the nucleotide sequence encoding a mutant phage lambda integrase.

In various embodiments, the E. coli strain of (i) further comprises a nucleotide sequence encoding for single chain integration host factor 2 (scIHF2). scIHF2 may have the amino acid sequence set forth in SEQ ID NO:9 or a functional variant or fragment thereof.

In various embodiments, IntC3 and scIHF2 are comprised in an expression cassette that is stably integrated into the genome of the E. coli strain. In such embodiments, the expression of both, IntC3 and scIHF2, may be stringently controlled by the same inducible expression control sequence, for example the endogenous arabinose operon. The expression cassette may comprise further elements, for example a selection marker, optionally flanked by recombination sites for later excision. Said recombination sites may be different from the mutant lambda integrase encoded by the expression cassette. In various embodiments, the expression cassette comprises the nucleotide sequence coding for IntC3, the nucleotide sequence coding for scIHF2, a nucleotide sequence coding for a selection marker, such as a chloramphenicol resistant gene, and two recombination sites flanking the selection marker, such as FIp recombinase recombination sites. In some embodiments, such expression cassette has the nucleotide sequence set forth in SEQ ID NO:1.

In various embodiments, the DNA sequence of interest comprises one or more genes. At least one of the one or more genes may be operably linked to expression control sequence(s).

In various embodiments, the second circular DNA construct comprising the DNA sequence of interest, i.e. the seamless DNA vector, does not contain bacterial sequences, with the exception of the phage lambda integrase recombination sequence. Said phage lambda integrase recombination sequence is an individual recombination sequence that is generated as a hybrid from the two recombination sites present in the bacterial plasmid as a result of the recombination.

In various embodiments, the two directly repeated lambda integrase recombination sequences that are recombination substrates for the mutant phage lambda integrase are selected from the group consisting of attP (SEQ ID NO:11) and attB (SEQ ID NO:12), attL (SEQ ID NO:13) and attB (SEQ ID NO:12), attL (SEQ ID NO:13) and attL (SEQ ID NO:13), as well as functional variants thereof. These functional variants are recombination competent. Functional variants of att sites may consist, without limitation, of pairs of attH4x (SEQ ID NO:14) and attP4x (SEQ ID NO:15), attL4x (SEQ ID NO:16) and attH4x (SEQ ID NO:14), attR4x (SEQ ID NO:17) and attH4x (SEQ ID NO:14), and attL4x (SEQ ID NO:16) and attR4x (SEQ ID NO:17).

The E. coli strain may be E. coli strain MG1655. The parental, unmodified E. coli strain MG1655 is a close derivative of the wild-type K12 strain, and was derived in 1981 [Guyer, M. S., R. E. Reed, T. Steitz, K. B. Low 1981. Cold Spr. Harb. Symp. Quant. Biol. 45:135-140]. Said strain is then engineered to comprise a nucleotide sequence encoding a mutant phage lambda integrase (IntC3) having the amino acid sequence set forth in SEQ ID NO:2 or a functional variant or fragment thereof, wherein the expression of said nucleotide sequence is stringently controlled by an inducible expression control sequence, to provide the strain of step (i).

In another aspect, the invention relates to an E. coli cell comprising a nucleotide sequence encoding a mutant phage lambda integrase (IntC3) having the amino acid sequence set forth in SEQ ID NO:2 or a functional variant or fragment thereof stably integrated into its genome, wherein the expression of said nucleotide sequence is stringently controlled by a genomic inducible expression control sequence.

In such cells, the nucleotide sequence encoding a mutant phage lambda integrase (IntC3) may be inserted into the genomic arabinose operon of E. coli. Integration may be immediately downstream of the arabinose promoter, for example by using the start codon of the endogenous araB gene as the start codon for the nucleotide sequence encoding a mutant phage lambda integrase.

The E. coli cell of the invention may further comprise a nucleotide sequence encoding for single chain integration host factor 2 (scIHF2) stably integrated into its genome. scIHF2 may have the amino acid sequence set forth in SEQ ID NO:9 or a functional variant or fragment thereof. In some embodiments, IntC3 and scIHF2 are comprised in an expression cassette that is stably integrated into the genome of the E. coli strain and wherein, optionally, the expression of both, IntC3 and scIHF2, is stringently controlled by the same inducible expression control sequence.

The E. coli cell may be derived from E. coli strain MG1655.

In various embodiments, the E. coli cell is obtainable by stably integrating a nucleotide sequence encoding a mutant phage lambda integrase (IntC3) having the amino acid sequence set forth in SEQ ID NO:2 or a functional variant or fragment thereof into the genome of an E. coli cell, for example an E. coli strain MG1655 cell, such the expression of said nucleotide sequence is stringently controlled by a genomic inducible expression control sequence.

### BRIEF DESCRIPTION OF THE DRAWINGS

The invention will be better understood with reference to the detailed description when considered in conjunction with the non-limiting examples and the accompanying drawings.

FIG. 1 shows a schematic diagram of the targeted arabinose operon in the E. coli genome for tightly regulated IntC3 expression as well as the structure of the ISC expression cassette, the nucleotide sequence of which is set forth in SEQ ID NO:1. The gene sequence shown in the lower part of the figure is set forth in SEQ ID Nos 21 and 22.
INTC3: integrase variant C3
IHF: single chain integration host factor 2
FRT: recombination site for FIp recombinase
CAT: chloramphenicol resistance cassette

The arrow at the bottom panel demarcates the position of ISC cassette insertion site at the start codon via homologous recombination.

FIG. 2 shows the analysis of junction PCRs and genomic PCR products by agarose gel electrophoresis of the arabinose operon after targeting as described in Example 1.

FIG. 3 shows the growth curve of E. coli MG1655 cells that have been transformed with either an expression cassette with IntC3 and scIHF2 encoding sequence (left curve) or the same expression cassette without the scIHF2 encoding sequence (right curve). “CAT” refers to the chloramphenicol resistance gene included as a selection marker.

FIG. 4 schematically shows the steps of multi-scale seamless vector production using the E. coli MG1655 strain transformed with IntC3 and scIHF2 as described herein. “att1” and “att2” refer to the recombinase sites and “at/t” refers to the hybrid recombination site generated by the recombination event. “payload” refers to the DNA sequence of interest.

FIG. 5 shows a vector map of attPhae2 (attL).

FIG. 6 shows the results of an agarose gel electrophoresis analysis of the episomal DNA isolated and subjected to different restriction enzymes (lanes: 1—substrate attLPhae2 undigested; 2—induced attPLPhae 2 undigested; substrate NdeI digested; 4—substrate ScaI digested; 5—induced, purified (NdeI+Exo); 3 out of 100 μl; 6—same as 5, but 6 out of 100 μl; 7—same as 5, but ScaI digest only (6 out of 100 μl); M—marker lanes).

FIG. 7 shows a vector map of the master plasmid attP4x attH4x with ClaI and EcoRV restriction sites.

FIG. 8 shows the result of an agarose gel electrophoresis analysis of the episomal DNA isolated and subjected to different restriction enzymes (lanes: 1: 1 kb Marker; 2: F8FI-Hygro Original 300 ng Digested with EcoRV; 3: ISC-F8FL-Hygro Uninduced 300 ng Digested with EcoRV; 4: Empty; 5: ISC-F8FL-Hygro Induced 10 ul Digested with EcoRV; 6: ISC-F8FI-Hygro Induced 10 ul Digested with CIa I; 7: ISC-F8FI-Hygro Induced 10 ul Digested with CIa I and 1 ul T5 Exonuclease; 8: Empty lane; 9: 100 bp Marker 3 ul.). The white arrow points at the seamless vector monomer.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail.

The present application provides a novel and versatile technology for multi-scale production of seamless DNA vectors that utilizes a mutant phage lambda integrase gene stably integrated into a tightly controlled region of a host cell's genome for integrase expression and subsequent site-specific DNA recombination using a suitable substrate plasmid.

In one aspect, the present application relates to methods for the in vivo production of seamless DNA vectors in E. coli, said seamless DNA vectors comprising a DNA sequence of interest and a phage lambda integrase recombination sequence.

The term “DNA sequence of interest” as used herein refers to any DNA sequence, the manipulation of which may be deemed desirable for any reason (e.g., conferring improved qualities and/or quantities, expression of a protein of interest in a host cell, expression of a ribozyme), by one of ordinary skill in the art. Such DNA sequences include, but are not limited to, coding sequences of structural genes (e.g., reporter genes, selection marker genes, oncogenes, drug resistance genes, growth factor genes), and non-coding sequences which do not encode an mRNA or protein product (e.g., promoter sequences, polyadenylation sequences, termination sequences, enhancer sequences, small interfering RNAs, short hairpin RNAs, antisense RNAs, microRNAs, long non-coding RNAs).

The DNA sequence of interest may comprise one or more genes, which may or may not be operably linked to one or more expression control sequences, such as a promoter, an enhancer, an operator, a termination signal, a 3′-UTR, or a 5′-UTR, an insulator. The term “operably linked” as used herein refers to the relationship between two or more nucleotide sequences that interact physically or functionally. For example, a promoter or regulatory nucleotide sequence is said to be operably linked to a nucleotide sequence that codes for an RNA or a protein if the two sequences are situated such that the regulatory nucleotide sequence will affect the expression level of the coding or structural nucleotide sequence.

In certain embodiments, the DNA sequence of interest may comprise a selection marker gene. The term “selection marker gene” as used herein refers to a gene that only allows cells carrying the gene to be specifically selected for or against in the presence of a corresponding selection agent. For example, selectable genes commonly used with eukaryotic cells include the genes for aminoglycoside phosphotransferase (APH), hygromycin phosphotransferase (HYG), dihydrofolate reductase (DHFR), thymidine kinase (TK), glutamine synthetase, asparagine synthetase, and genes encoding resistance to neomycin (G418), puromycin, histidinol D, bleomycin and phleomycin. Selection markers that are used for prokaryotic cells to select for cells that have been successfully transformed, will be described below.

The DNA sequence of interest that forms part of a seamless vector may be designed for stable integration into a target genomic sequence of a host cell, such as a eukaryotic cell. The term “stably integrating a DNA sequence of interest into a target genomic DNA sequence of a host cell”, as used herein, refers to the stable integration of the DNA sequence of interest into the nuclear genome or any other extranuclear genomic material within a cellular compartment of interest, e.g. a mitochondrion, by forming covalent bonds with the host DNA. The stably integrated DNA sequence of interest will thus be heritable to the progeny of a thus modified host cell. Said stable integration may be performed in all types of cells in vitro, ex vivo, or in vivo. The host cell may be a eukaryotic cell, preferably a mammalian cell, more preferably a human cell. For example, the host cell may be a bacterial cell, a yeast cell, a plant cell, or a human cell; it may be a cancer cell, an oocyte, an embryonic stem cell, a hematopoietic stem cell, or any type of differentiated cells.

The methods described herein comprise the step of providing an E. coli strain comprising a nucleotide sequence encoding a mutant phage lambda integrase (IntC3) having the amino acid sequence set forth in SEQ ID NO:2 or a functional variant or fragment thereof, wherein the expression of said nucleotide sequence is stringently controlled by an inducible expression control sequence.

The term “phage lambda integrase” as used herein refers to any phage lambda-derived integrases that possess endonuclease and ligase activities. As known in the art, the phage lambda integrase like Cre and FIp belongs to the integrase family of the sequence-specific conservative DNA recombinases and catalyses the integrative recombination between two different recombination att sites.

The integrase used in the method of the present invention is a specific mutant of phage lambda integrase known in the art, namely the one disclosed WO2016022075A1, which hereby incorporated by reference, and termed “IntC3”. Said IntC3 mutant integrase has the amino acid sequence set forth in SEQ ID NO:2. The DNA sequence encoding said mutant integrase may have the nucleotide sequence set forth in SEQ ID NO:10.

The term “functional variant”, as used herein in relation to the integrase, relates to integrases that differ from the amino acid sequence set forth in SEQ ID NO:2 by one or more amino acid substitutions, additions or deletions but retain the functionality of the reference sequence. In such variants the amino acid positions that define the reference integrase C3, namely the positions 43F, 319G, and 336V may be invariable. The term also encompasses variants that comprise the sequence set forth in SEQ ID NO:2 but comprise N- and/or C-terminal extensions of 1 or more amino acids. Generally, the term “variant” covers such integrases that have at least 90% sequence identity with the sequence set forth in SEQ ID NO:2 over their entire length, preferably at least 91, 92, 93, 94, 95, 96, 97, 98, or 99% sequence identity. In these variants, the positions 43F, 319G, and 336V may still be invariable. The identity of nucleic acid sequences or amino acid sequences is generally determined by means of a sequence comparison. This sequence comparison is based on the BLAST algorithm that is established in the existing art and commonly used (cf. e.g. Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

The term “functional fragment” or “fragment”, as used herein in relation to the integrase, relates to integrases that differ from the amino acid sequence set forth in SEQ ID NO:2 by a deletion of one or more amino acids from its C- and/or N-terminus. Said fragments preferably retain full functionality. In various embodiments, such fragment differs from the reference sequence and that they lack 1-20 amino acids from their N- and/or C-terminus, for example 1-15 amino acids or 1-10 amino acids or 1-5 amino acids.

The mutant integrases disclosed herein are, in contrast to the wild-type integrase, able to perform the recombination reaction without a co-factor, such as IHF. However, as described below, the addition of the co-factor gene, in particular of scIHF2 (SEQ ID NO:9), can have beneficial effects in that it seems to substantially reduces the lag phase in cultivation and thus can shorten incubation times needed to reach the desired cell density.

The inducible expression control sequence may be the E. coli arabinose operon or any other suitable stringently controlled and inducible gene control element present in the E. coli genome. In embodiments where the arabinose operon is used, the induction in step (iv) may be triggered by the addition of arabinose or any arabinose derivative or mimic that can also induce the operon. In such embodiments, the nucleotide sequence encoding a mutant phage lambda integrase (IntC3) may be inserted into the genomic arabinose operon of E. coli, downstream of the arabinose promoter, for example immediately downstream of the promoter, for example by using the start codon of the endogenous araB gene, which is the first endogenous gene downstream of the promoter sequence, as the start codon for the nucleotide sequence encoding IntC3. If an expression cassette is used, the whole cassette may be inserted accordingly.

The method further comprises the step of transforming a bacterial plasmid comprising a DNA sequence of interest and a bacterial backbone sequence flanked by two directly repeated lambda integrase recombination sequences that are recombination substrates for the mutant phage lambda integrase into the E. coli strain of (i), wherein the bacterial backbone sequence typically comprises a selection marker.

The term “bacterial plasmid” as used herein refers to a circular DNA molecule capable of replication in a bacterial host cell. A bacterial plasmid may contain an appropriate origin of replication, which is a sequence of DNA sufficient to enable the replication of the plasmid in a host bacterial cell. A bacterial plasmid may also contain a selectable marker sequence, which encodes a selectable marker conferring cellular resistance to antibiotics such as ampicillin, kanamycin, chloramphenicol, and tetracycline. In preferred embodiments, the bacterial plasmid is negatively (−) supercoiled.

The lambda integrase recombination sequences that are recombination substrates for the mutant phage lambda integrase are pairs of att site derivatives. An att sequence is the recognition site where binding, cleavage, and strand exchange are performed by the phage lambda integrase and any associated accessory proteins thereof. These att sites may be selected from the group consisting of the pairs of attP (SEQ ID NO:11) and attB (SEQ ID NO:12), attL (SEQ ID NO:13) and attB (SEQ ID NO:12), attL (SEQ ID NO:13) and attL (SEQ ID NO:13). Furthermore, functional variants of att sites consisting of pairs of attH4x (SEQ ID NO:14) and attP4x (SEQ ID NO:15), attL4x (SEQ ID NO:16) and attH4x (SEQ ID NO:14), attR4x (SEQ ID NO:17) and attH4x (SEQ ID NO:14), and attL4x (SEQ ID NO:16) and attR4x (SEQ ID NO:17) may also be selected.

The term “directly repeated orientation” as used herein indicates that the recombination sites in a set of recombinogenic recombination sites are arranged in the same orientation (e.g., 5′ to 3′), such that the recombination between these sites results in excision, rather than inversion, of the intervening DNA sequence. The term “inverted orientation” as used herein indicates that the recombination sites in a set of recombinogenic recombination sites are arranged in the opposite orientation, so that the recombination between these sites results in inversion, rather than excision, of the intervening DNA sequence. Therefore, for the successful implementation of the intramolecular recombination of step (ii) as described herein, the two recognition sites flanking the DNA sequence of interest as described herein are arranged in a directly repeated orientation.

If integrase-mediated recombination occurs between two compatible recognition sites that are on the same molecule, the intramolecular recombination results in either the deletion or inversion of a sequence flanked by the two recognition sites. More specifically, when two recognition sites on the same DNA molecule are in a directly repeated orientation, integrase excises the DNA between these two sites leaving a single recognition site on the DNA molecule; if two recognition sites are in inverted orientation on a single DNA molecule, integrase inverts the DNA sequence between these two sites rather than removing the sequence.

The method further comprises the step of cultivating the transformed E. coli cells under conditions selective for the selection marker comprised in the bacterial plasmid. This ensures that only those cells that have been successfully transformed are grown. It is further important that in this cultivation step, the integrase is not yet expressed, as any leaky expression of the integrase at this stage will lead to premature loss of the seamless vector inside the bacterium and thus severely comprised vector yield. As this cultivation step is necessary to grow the cells and thus increase the copy number of the bacterial plasmid, the methods of the invention require that the integrase is under the control of an inducible yet stringent expression control element.

It has been found by the inventors that if the E. coli strain further comprises a nucleotide sequence encoding for single chain integration host factor 2 (scIHF2), the lag phase and the time to reach stationary phase with/without induction may be significantly shortened. i.e. the time to reach the desired cell densities is significantly reduced. Therefore, the strain used may also comprise the scIHF2 coding sequence, for example that having the amino acid sequence set forth in SEQ ID NO:9 or a functional variant or fragment thereof. The variants and fragments are defined as those of the integrase described above (with the exception of the mutants specific for the integrase). In the utilized strain, IntC3 and scIHF2 may even be comprised in a single expression cassette that is stably integrated into the genome of the E. coli strain. In such embodiments, the expression of both, IntC3 and scIHF2, may thus be stringently controlled by the same inducible expression control sequence, for example the endogenous arabinose operon. The expression cassette may comprise further elements, for example a selection marker, optionally flanked by recombination sites for later excision. Said recombination sites may be different from the mutant lambda integrase encoded by the expression cassette. In various embodiments, the expression cassette comprises the nucleotide sequence coding for IntC3 (SEQ ID NO:10), the nucleotide sequence coding for scIHF2 (SEQ ID NO:18), a nucleotide sequence coding for a selection marker, such as a chloramphenicol resistant gene (SEQ ID NO:19), and two recombination sites flanking the selection marker, such as FIp recombinase recombination sites (SEQ ID NO:20). In some embodiments, such expression cassette has the nucleotide sequence set forth in SEQ ID NO:1. These recombination sites allow the targeted excision of the genomic selection marker at a later stage of the method. The recombination sites are therefore preferably in directly repeated orientation.

In the next step of the method, once a desired cell density has been reached (e.g. OD600=0.5), the expression of the mutant phage lambda integrase is induced. Said induction can be done by adding an agent or compound that induces the expression control sequence for the integrase coding sequence.

The expression of the integrase leads to the presence of both, the integrase and the bacterial plasmid in the bacterial host cell. The contact of the two facilitates intramolecular recombination of the two directly repeated lambda integrase recombination sequences in the bacterial plasmid. Said excision and recombination event yields a dimeric DNA catenane consisting of a first circular DNA molecule that carries the bacterial backbone and a second circular DNA molecule that carries the DNA sequence of interest and a phage lambda integrase recombination sequence that is a hybrid of the two directly repeated lambda integrase recombination sequences. Such catenane rings are generally described in Spengler et al. (“The stereostructure of knots and catenanes produced by phage lambda integrative recombination: implications for mechanism and DNA structure”, Cell, vol. 42, No. 1, August 1985, 325-334).

By means of the “intramolecular recombination” between the two lambda integrase recognition sites flanking the bacterial backbone sequence (and the DNA sequence of interest), as mediated by the integrase, obtained are a first circular DNA construct comprising or essentially consisting of the bacterial backbone of the plasmid that contains essentially all bacterial DNA elements, such as those described above, that are used for multiplication in the bacterial host cell, and a second circular DNA construct that comprises or essentially consists of the DNA sequence of interest and a nucleotide sequence that arises from the recombination event and is a hybrid of the two directly repeated lambda integrase recombination sequences.

The term “essentially consisting of”, as used herein in this context, is a partially open term, which does not exclude additional, unrecited element(s), step(s), or ingredient(s), as long as these additional element(s), step(s) or ingredient(s) do not materially affect the basic and novel properties of the invention. Said term refers to that the second circular DNA construct consists of the DNA sequence of interest, the nucleotide sequence that arises from the recombination event and is a hybrid of the two directly repeated lambda integrase recombination sequences, and the nucleotide stretches originally present between the DNA sequence of interest and the two flanking sequences. In preferred embodiments, said nucleotide stretches on each side of the DNA sequence of interest have up to 1,000 nt, preferably up to 500 nt, and more preferably up to 100 nt in length. It is preferred that said nucleotide stretches do not make a significant portion (e.g. less than 1%, 5%, or 10%) of the whole construct. In some embodiments, the DNA sequence of interest in the bacterial plasmid is immediately flanked by the two directly repeated lambda integrase recombination sequences, in which case such nucleotide stretches are absent and the resultant second circular DNA construct does not contain bacterial sequences, except the nucleotide sequence that arises from the recombination event of the two recombination sites. In some embodiments, the bacterial plasmid of the present application is designed with minimized nucleotide stretches flanking the DNA sequence of interest insofar as the subsequent intramolecular recombination and the genomic integration of the DNA sequence of interest are not significantly and adversely affected.

Without wishing to be bound to any particular theory, it is believed that intramolecular recombination is thermodynamically strongly favored over intermolecular recombination; hence, under standard reaction conditions, intermolecular recombination is a minor byproduct and even if it occurred, can be clearly distinguished from intramolecular recombination due to molecular size differences. However, the relative concentrations of the bacterial plasmid and the integrase as well as the various parameters of the reaction condition can still be optimized by routine experimentation to favor intramolecular recombination over intermolecular recombination between two different bacterial plasmids.

As a consequence of this reaction, the second circular DNA molecule which constitutes the seamless DNA vector can be a mini circular plasmid devoid of or essentially devoid of bacterial sequences except the nucleotide sequence is a hybrid of the two recombination sites used for the excision. Such mini circular plasmids provide superior alternatives to traditional plasmids. They exhibit better bioavailability compared to conventional plasmids due to their smaller size, and improved immuno-compatibility due to the reduction or elimination of undesired bacterial sequences. In addition, their smaller size may also confer higher delivery efficiency and lower toxicity. Alternatively, the method may similarly be used to generate longer circular plasmids, since the length of the plasmid is not limited. The examples comprised herein show that the methods described can be effectively used for the production of both mini and maxi plasmid vectors.

As the product is a catenane, this step may comprise unlinking the two catenated circular DNA molecules. In some embodiments, the first circular DNA construct comprising the bacterial sequences may be linearized by means of an endonuclease activity, preferably by means of a restriction enzyme, while leaving the second circular DNA construct intact. Such a digestion step may also serve to digest damaged, such as nicked, circular molecules. The second circular DNA construct is then further isolated. The choice of the endonuclease and the isolation method is within the knowledge of the person of average skill in the art.

The isolation of the seamless DNA vector may be done by any suitable means. Typically, this involves lysing the cells or breaking the cell wall by suitable means, such as ultrasound, homogenization, French press, etc. and subsequent separation of all undesired cellular debris, for example by centrifugation and/or filtration. The DNA vectors may then be separated by solvent extraction or chromatography techniques, all of which are known to those skilled in the art.

The E. coli strain used in the inventive methods may be E. coli strain MG1655. The parental, unmodified E. coli strain MG1655 has been described in the art [Guyer, M. S., R. E. Reed, T. Steitz, K. B. Low 1981. Cold Spr. Harb. Symp. Quant. Biol. 45:135-140] and has been extensively used in the field. According to the present invention said strain is engineered to comprise a nucleotide sequence encoding the mutant phage lambda integrase (IntC3) described herein under the stringent control of an endogenous expression control element.

The seamless DNA vectors comprising the DNA sequence of interest may, after isolation, be used for introduction into another host cell by any means available in the art, including but not limited to DNA transfection, biolistic technology, ultrasound, nanoparticles, or microinjection. In said host cells, the DNA sequence of interest may be integrated into the host cell's genome by appropriate means and techniques, such as those described in US 2017/0362606 A1.

The invention also pertains to the E. coli cells described herein. These have been modified such that they comprise a nucleotide sequence encoding a mutant phage lambda integrase (IntC3) having the amino acid sequence set forth in SEQ ID NO:2 or a functional variant or fragment thereof stably integrated into its genome, wherein the expression of said nucleotide sequence is stringently controlled by a genomic inducible expression control sequence.

All embodiments disclosed above in relation to the methods of the invention similarly apply to the cells of the invention and vice versa.

These cells may then be used in the described methods or may be generally used for the production of seamless DNA vectors from the bacterial plasmids described herein—Such uses thus also form part of the present invention.

The present invention is further illustrated by the following examples. However, it should be understood, that the invention is not limited to the exemplified embodiments.

### EXAMPLES

### Example 1: Engineering of Escherichia coli (E. coli) Strain MG1655 Carrying an Inducible a Integrase C3 Expression Cassette

The E. coli strain MG1655 was chosen as a base for generating a versatile seamless vector bacterial producer strain because it approximates K12 wild-type cells with minimal prior genetic changes [Blattner F R, et al (1997) Science. 277(5331):1453-62]. This feature is assumed to provide for higher chances of success for tightly regulated expression of enhanced phage λ integrase variant IntC3 via the endogenous arabinose operon, which is a prerequisite for seamless vector production in vivo. The E. coli genome project (https://www.genome.wisc.edu/resources/strains.htm) states with respect to MG1655 that this strain approximates wild-type E. coli and has been maintained as a laboratory strain with minimal genetic manipulation, i.e. having only been cured of the temperate bacteriophage lambda and F plasmid by means of ultraviolet light and acridine orange, respectively. The MG1655 strain was originally derived by Mark Guyer from strain W1485, which, in turn, was derived from a stab-culture descendant of the original K-12 isolate [Guyer, M. S., R. E. Reed, T. Steitz, K. B. Low 1981. Cold Spr. Harb. Symp. Quant. Biol. 45:135-140]. The original E. coli strain K-12 was obtained from a stool sample of a diphtheria patient in Palo Alto, CA in 1922 [Bachmann, B., pp. 2460-2488 in Neidhardt et al. (1996), Escherichia coli and Salmonella: Cellular and Molecular Biology, ASM Press’].

The multi-transgene deoxyribonucleotide (DNA) sequence set forth in SEQ ID NO:1, termed ISC, was commercially synthesized by the company GeneScript: IntC3_scIHF2_FRT_CAT_FRT_LexA (2725 bp) (see FIG. 1; IntC3: integrase variant C3 (SEQ ID NO:2); IHF: single chain integration host factor 2; FRT: recombination site for FIp recombinase; CAT: chloramphenicol resistance cassette).

The chosen strategy for MG1655 engineering included the precise insertion of the ISC expression cassette into the genomic arabinose operon of MG1655 immediately downstream of the arabinose promoter by using the start codon of the endogenous araB gene as the start codon for IntC3 (FIG. 1). Two primers were designed to insert the construct at this locus using routine protocols of lambda red-mediated homologous recombination reactions inside MG1655 cells [Thomason, L., D. L. Court, M. Bubunenko, N. Costantino, H. Wilson et al., 2007 Curr. Protoc. Mol. Biol. 78:1.16.1-1.16.24]. The ATG start codon of the IntC3 gene was re-introduced in the forward primer. PCR amplification of the ISC construct for electroporation into electroporation-competent MG1655 cells was performed with the following primers:

IntC3_ARAB_FWD_HR:
(SEQ ID NO: 3)
ACTCTCTACTGTTTCTCCATACCCGTTTTTTTGGATGGAGTGAAACG
ATGGGAAGAAGGCGAAGTCATGAGC
FRT_ARAB_REV_HR:
(SEQ ID NO: 4)
GCCAAAGCTCGCACAGAATCACTGCCAAAATCGAGGCCAATTGCAAT
CGCTTATACAGTCGAAGTTCCTATA

### PCR Reaction Conditions:

REACTION

| COMPONENT | 50 μl REACTION | |
|---|---|---|
| 5X Q5 Reaction Buffer | 10 | μl |
| 10 mM dNTPs | 1 | μl |
| 10 μM Forward Primer | 2.5 | μl |
| 10 μM Reverse Primer | 2.5 | μl |
| Template plasmid | 10 | ng |
| Q5 High-Fidelity DNA | 0.5 | μl |
| Polymerase | | |
| Nuclease-Free Water | to 50 | μl |

| PCR parameters | Q5 High-Fidelity DNA Polymerase (NEB) |
|---|---|
| Initial denaturation | 98° C. for 30 seconds |
| Denaturation | 98° C. for 10 seconds |
| Annealing | 65° C. for 30 seconds |
| Extension | 72° C. for 30 seconds/1 kb |
| Final extension | 72° C. for 2 min |
| Hold | 4° C. |

The resulting PCR product was analyzed through agarose gel electrophoresis, gel purified and stored for subsequent electroporation into MG1655/pKD46 electroporation competent cells.

MG1655 cells were grown on an agar plate and a single colony was inoculated for an overnight culture. Cells were made competent for plasmid transformation using standard protocols. Plasmid pKD46 [Datsenko, K A, B L Wanner (2000) Proc. Natl. Acad. Sci. U.S.A. 97(12):6640-5] was transformed, cells plated on selection media, and grown at 30° C. overnight.

One colony of MG1655 carrying pKD46 was grown overnight in DYT media AMP⁺²⁰⁰ at 30° C. and shaking at 180 rpm. The stationary culture was diluted 1:200 in fresh 200 ml DYT media and incubated at 30° C., with shaking at 180 rpm. OD was determined at 600 nm and when it had reached 0.4, the culture was induced with 1.2% L-arabinose (16 ml of 15% arabinose stock) and incubated at 37° C. for 1 hr at 180 rpm. The flask was then immediately placed into an ice water bath. The culture was left on ice for 20 minutes with occasional agitation; from this moment on, cells were always kept chilled. Cells were distributed into 4×50 ml Falcon tubes on ice and kept on ice. Centrifugation was at 1000 g for 10 to 20 min at 4 degrees; the supernatant must be clear and the pellet must be visible. In case, the supernatant is turbid, the centrifugation time must be expanded. Once the supernatant is clear, the supernatant is discarded and pellets suspended in 25 ml of double-deionized ice-cold water. Contents of 2×50 ml Falcon tubes are combined and centrifuged again under the same conditions. After the spin, if the supernatant is clear, each pellet is suspended in 50 ml ice cold water and centrifugation is repeated. Supernatant is discarded and pellet suspended in 50 ml ice cold 10% glycerol (diluted in DD water). The centrifugation is repeated and the supernatant discarded. The pellet is resuspended in 0.5 ml 10% glycerol. The two re-suspended pellets are combined in 2 ml pre-chilled Eppendorf tubes and mixed. OD₆₀₀ of a 1:100 diluted solution (10 μl suspended cells+990 μl of 10% glycerol) should be 0.4 to 0.6. Competent cells are stored in aliquots of 70 μl-100 μl per tube on dry ice. Flash freeze with liquid nitrogen and store at minus 80 degrees.

Electroporation of the ISC construct into competent MG1655/pKD46 cells was performed with Gene Pulser (BioRad) as follows: 1 to 10 ng of the ISC PCR product were added to 100 μl competent E. coli and electroporated in pre-set conditions (set 1 or 2). Cell recovery was at 37° C. for 1 hr in DYT without antibiotics. The transformed cells were spread onto DYT media+0.1% Glucose+15 μg/ml chloramphenicol agar plates and grown at 30° C. Growth at 37° C. will subsequently lead to the loss of pKD46 plasmid since it carries a temperature sensitive origin of replication.

Resulting colonies were tested by colony PCR to confirm the left and right junctions of the genomic inserted transgene cassette as well as the presence of the entire cassette by genomic PCR.

Primers used for PCRs:
Left Junction: PCR product = 291 bp
ARAC_FWD:
(SEQ ID NO: 5)
GTCTATAATCACGGCAGAAAAGTCC
IntC3_REV:
(SEQ ID NO: 6)
TCGCCTGTCTCTGCCTAATCC
Right Junction: PCR product = 397 bp
CAT_FWD:
(SEQ ID NO: 7)
CGCAAGGCGACAAGGTGCT
ARAB REV:
(SEQ ID NO: 8)
CCGCTTCCATTGACTCAATGTAGTC
Genomic PCR (2.85 kb):
ARAC_FWD + ARAB REV

### Colony PCR:

One colony: diluted in 50 μl DYT media+0.1% Glucose+15 μg/ml chloramphenicol media: 2 μl used as colony PCR template.

### PCR Reaction:

| COMPONENT | 25 μl REACTION | |
|---|---|---|
| GoTaq ® Reaction Buffer | 5 | μl |
| 25 mM MgCl2 Solution | 1.5 | μl |
| 10 mM dNTPs | 0.5 | μl |
| 10 μM Forward Primer | 0.5 | μl |
| 10 μM Reverse Primer | 0.5 | μl |
| Template DNA/colony | 2 | μl |
| GoTaq@ DNA Polymerase | 0.2 | μl |
| Nuclease-Free Water | to 25 | μl |

| PCR parameters | GoTaq Flexi DNA polymerase (Promega) |
|---|---|
| Initial denaturation | 95° C. for 5 min |
| Denaturation | 95° C. for 1 min |
| Annealing | 56° C. for 30 seconds |
| Extension | 72° C. for 1 minute/1 kb |
| Final extension | 72° C. for 5 min |
| Hold | 4° C. |

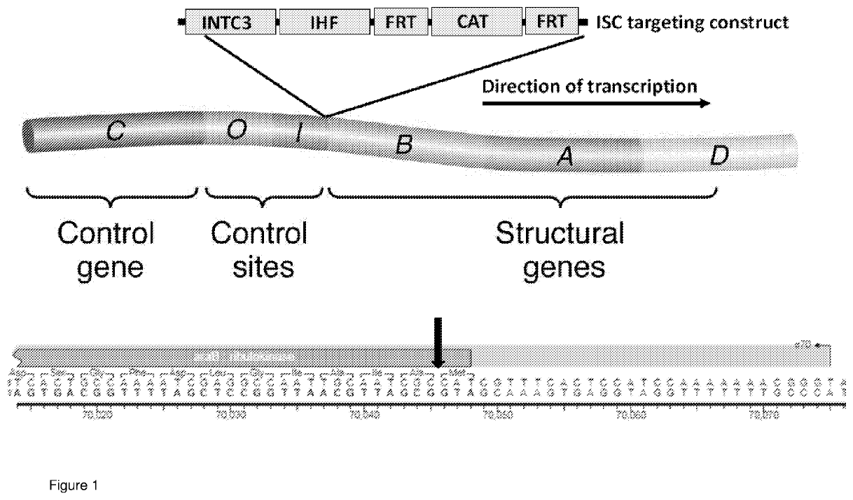

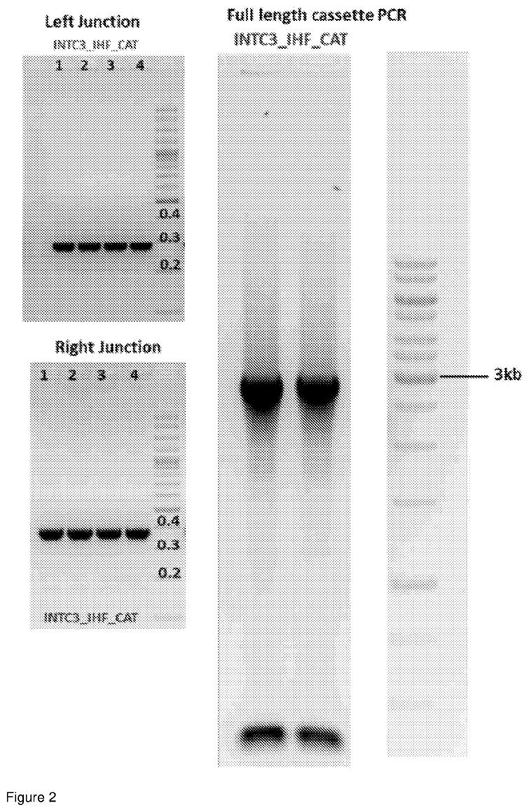

Junction PCR and genomic PCR products were analyzed by agarose gel electrophoresis as shown in FIG. 2. The analyzed clones were positive for both transgene junctions as well as the full-length genomic insertion. PCR products were verified by sequencing, which revealed a complete match with the predicted genome/construct sequences. We dubbed our engineered new strain as MG1655-ISC. At this point, we retained the CAT gene as part of the transgene cassette. By including the FRT sequences for the yeast FIp recombinase in our construct (FIG. 1), the option remains to remove the CAT expression cassette by transient FIp recombinase expression vector at a later stage.

### Example 2: Seamless Vector Production Using Engineered E. coli Strain MG1655-ISC

A critical parameter of any engineered E. coli strain for seamless vector production is the cell doubling time. Growth rates of MG1655-ISC were analyzed by measuring the optical density at 600 nm, and it was compared with that of a variant MG1655 strain that carries the same transgene expression cassette at the arabinose locus, except for the gene coding for scIHF2. The latter strain was dubbed MG1655-IC and was generated in parallel to MG1655-ISC following the same protocol except for the use of a different transgene targeting PCR construct.

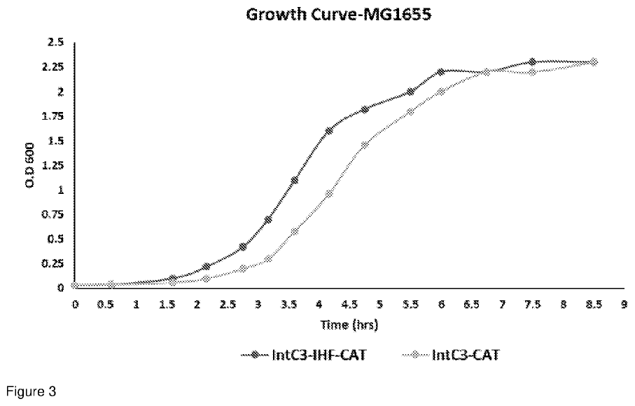

An example of the growth rate analysis is shown in FIG. 3 and revealed that the presence of the scIHF2 gene sequence in the genome leads to a substantial shortening of the time to reach desired cell densities. This represents an advantage for large scale seamless vector production because it shortens incubation time to reach the desired cell density for induction of IntC3 expression by arabinose.

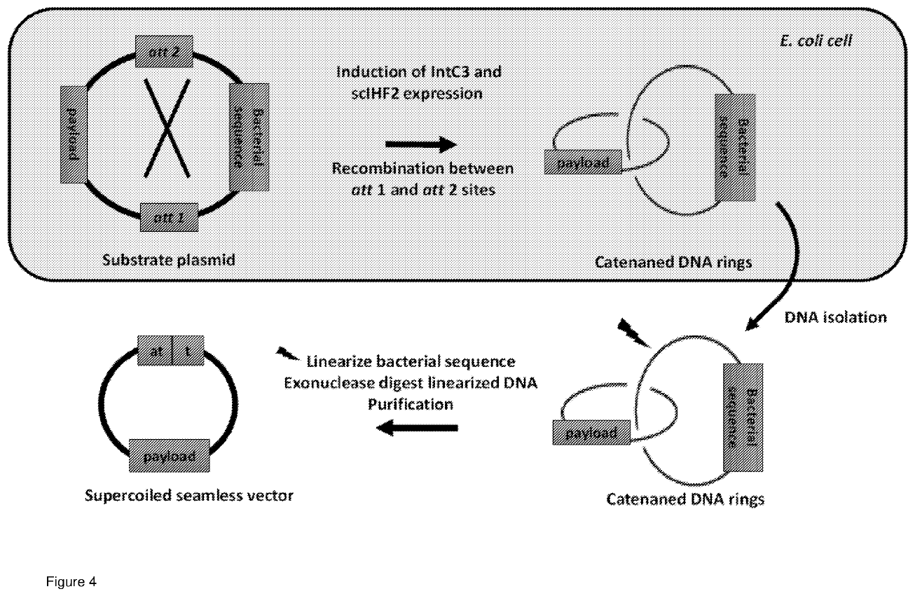

The general workflow of seamless vector production using strain MG1655-ISC is shown in FIG. 4. Briefly, a plasmid with a standard bacterial backbone, which is flanked by two directly repeated lambda integrase recombination sequences, termed att1 and att2, and carrying the desired DNA payload for seamless vector production is transformed into MG1655-ISC by routine bacterial transformation and selection. The transformed cells are grown in liquid LB culture medium with antibiotics until OD₍₆₀₀₎ reaches a value of approximately 1.0. At that time point, IntC3 and scIHF2 expression is induced by the addition of arabinose to the medium, and cells are incubated for additional 70-90 minutes at 37 degrees. Induction of IntC3 inside MG1655-ISC leads to recombination between att1 and att2, which generates a dimeric DNA catenane consisting of one DNA ring that carries the bacterial backbone and a second DNA ring that carries the DNA payload plus one copy of hybrid att (FIG. 4) The catenated DNA rings are unlinked inside E. coli by endogenous type 2 topoisomerases leading to two separate DNA rings (not depicted in FIG. 4).

Following induction of IntC3 and scIHF2 transgene expression by arabinose, episomal DNA is purified from lysed MG1655-ISC cells by standard procedures, and the bacterial DNA ring is linearized by restriction digest using suitable commercially available restriction enzymes which hydrolyze the DNA only in the bacterial backbone. The linearized DNA and contaminating nicked DNA molecules are subsequently digested by incubation with commercially available phage T5 exonuclease (FIG. 4, bottom half). The remaining intact covalently closed supercoiled seamless DNA vector can be purified by, for example, phenol/chloroform extraction, precipitated with alcohol and dissolved in a suitable solvent.

### Example 3: Mini-Seamless Vector Production Using Variants of attL and attB Recombination Sites in MG1655-ISC

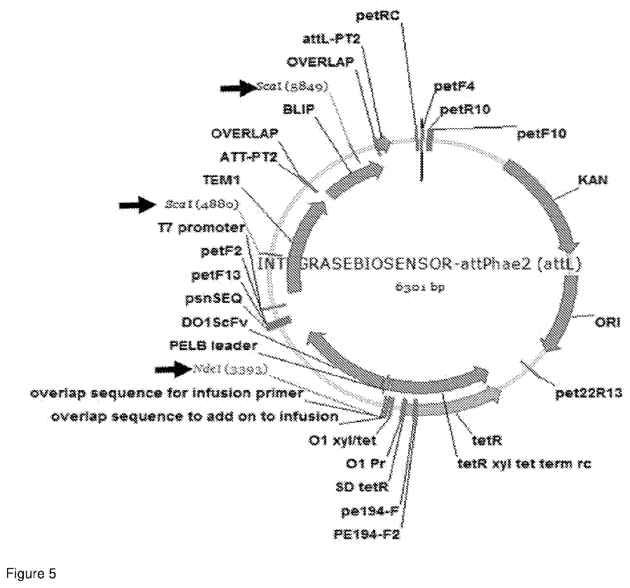

To demonstrate broad utility of MG1655-ISC for seamless vector production, substrate plasmid pattPhae2(attL) (FIG. 5) was transformed using standard protocols. This recombination substrate carries a 21 bp attB homologue and a 121 bp attL homologous sequence in direct orientation separated by about 500 bp. Recombination by IntC3 will result in two DNA circles: a small supercoiled 500 bp mini-seamless vector plus a hybrid attB sequence, and a 5.8 kb supercoiled DNA that carries the bacterial backbone and other sequences.

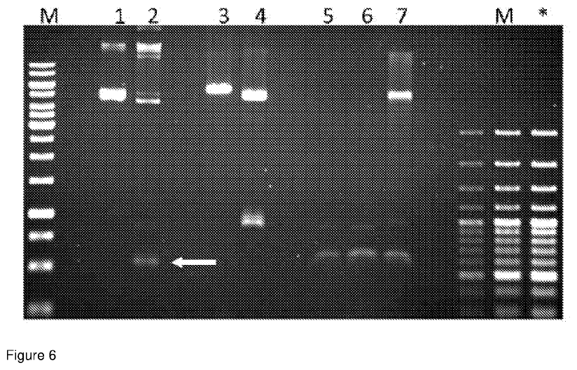

MG1655-ISC cells transformed with pattPhae2(attL) were induced by arabinose for 70 minutes and harvested. Episomal DNA was isolated and analyzed by agarose gel electrophoresis for recombination products. The results depicted in FIG. 6 revealed that compared to the substrate DNA (lane 1), induction of IntC3 expression produced more than 90% recombination products which were decatenated efficiently in vivo by endogenous topoisomerases, i.e. the two recombination product DNA rings were no longer topologically linked (lane 2). The released 500 bp supercoiled mini-seamless vector (lane 2, FIG. 6) migrates far ahead from the rest of the isolated DNA due to its small size. Restriction digest of substrate DNA and recombination products by either ScaI (arrows in the map in FIG. 5) confirmed that more than 90% of the substrate has been recombined inside MG1655-ISC cells after arabinose induction (lanes 4 and 7, FIG. 6). Restriction digest of DNA isolated from induced cells by NdeI, which cleaves only in the bacterial backbone segment, in the presence of exonuclease T5 (Exo) resulted in pure supercoiled seamless vector DNA (lanes 5 and 6, FIG. 6). The substrate DNA digested with NdeI without Exo treatment is shown as control in lane 3. It was determined that about 3 μg of pure supercoiled mini-seamless vector can easily be produced from a 100 ml culture.

### Example 4: Maxi-Seamless Vector Production Using Variants of attP and attB Recombination Sites in MG1655-ISC

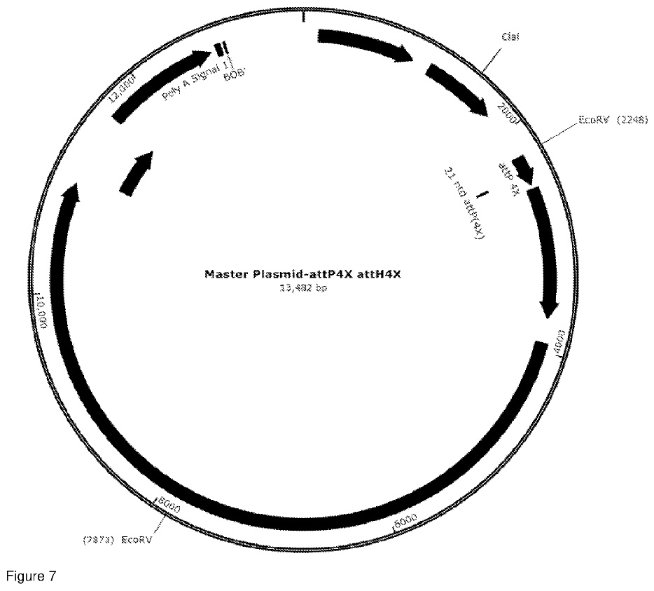

In another example demonstrating broad utility of strain MG1655-ISC for seamless vector production, substrate plasmid pEF1a-FLF8-Ires-Hygro was transformed (FIG. 7). This recombination substrate carries a 21 bp attB homologue (SEQ ID NO:14) and a 241 bp attP homologue (SEQ ID NO:15) sequence in direct orientation separated by about 3 kb. Recombination by IntC3 will result in a large supercoiled 10.4 kb seamless vector that carries a human blood clotting factor 8-Ires-hygromycin expression cassette (map in FIG. 7) plus a hybrid attL sequence. The second product is a 3 kb supercoiled DNA that carries the bacterial genetic elements plus a hybrid attR sequence.

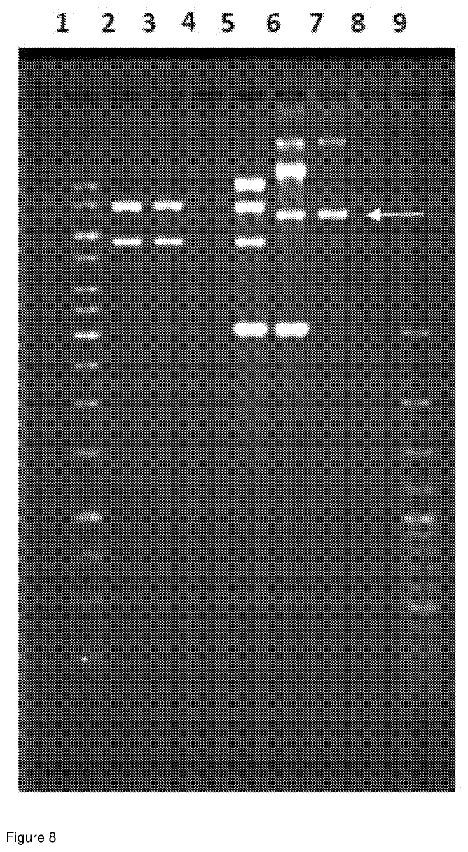

Transformed MG1655-ISC cells were induced by arabinose and harvested. Episomal plasmid DNA was isolated by standard procedures and analyzed by agarose gel electrophoresis for recombination products. Results obtained with EcoRV-digested DNA (FIG. 8) revealed that compared to the substrate DNA (lane 2), nearly 100% of the plasmid remained unrecombined before induction of IntC3 expression in vivo (lane 3), hence demonstrating the ultra-tight regulation of IntC3 gene in the engineered arabinose operon. Induction of IntC3 expression by arabinose produced more than 70% of the predicted 10.4 kb and 3 kb recombination products (EcoRV digest, lane 5). Restriction digest of recovered DNA after incubation with ClaI, which cleaves only in the bacterial DNA backbone (see map in FIG. 7), results in supercoiled 10.4 kb seamless vector, the linearized unrecombined substrate (13.4 kb) and the linearized bacterial backbone (3 kb) (lane 6). Moreover, the 10.4 kb seamless vector is the only remaining product after addition of exonuclease T5 to the C/al-digested DNA (lane 7). In this example, a fraction of seamless vector dimers was also observed, which are about 21 kb in size. It was determined that 60 to 90 μg of pure supercoiled maxi-seamless vector can be produced in a 100 ml culture.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. A method for the in vivo production of seamless DNA vectors in E. coli, said seamless DNA vectors comprising a DNA sequence of interest and a phage lambda integrase recombination sequence, the method comprising:
(i) providing an E. coli strain comprising a nucleotide sequence encoding a mutant phage lambda integrase (IntC3) having the amino acid sequence set forth in SEQ ID NO:2 or a functional variant or fragment thereof, wherein the expression of said nucleotide sequence is stringently controlled by an inducible expression control sequence;
(ii) transforming a bacterial plasmid comprising a DNA sequence of interest and a bacterial backbone sequence flanked by two directly repeated lambda integrase recombination sequences that are recombination substrates for the mutant phage lambda integrase into the E. coli strain of (i), wherein the bacterial backbone sequence comprises a selection marker;
(iii) cultivating the transformed E. coli cells under conditions selective for the selection marker comprised in the bacterial plasmid;
(iv) inducing the expression of the mutant phage lambda integrase to facilitate recombination of the two directly repeated lambda integrase recombination sequences in the bacterial plasmid to obtain a dimeric DNA catenane consisting of a first circular DNA molecule that carries the bacterial backbone and a second circular DNA molecule that carries the DNA sequence of interest and a phage lambda integrase recombination sequence that is a hybrid of the two directly repeated lambda integrase recombination sequences; and
(v) isolating the second circular DNA molecule that carries the DNA sequence of interest and a phage lambda integrase recombination sequence.

2. The method of claim 1, wherein step (iv) comprises unlinking the two catenated circular DNA molecules, and/or step (v) comprises linearizing the first circular DNA molecule.

3. (canceled)

4. The method of claim 1, wherein the nucleotide sequence encoding a mutant phage lambda integrase having the amino acid sequence set forth in SEQ ID NO:2 or a functional variant or fragment thereof is stably integrated into the E. coli strains genome.

5. The method of claim 1, wherein the inducible expression control sequence is the E. coli arabinose operon and wherein, optionally, induction in step (iv) is triggered by the addition of arabinose.

6. The method of claim 5, wherein the nucleotide sequence encoding a mutant phage lambda integrase (IntC3) having the amino acid sequence set forth in SEQ ID NO:2 or a functional variant or fragment thereof is inserted into the genomic arabinose operon of E. coli immediately downstream of the arabinose promoter by using the start codon of the endogenous araB gene as the start codon for the nucleotide sequence encoding a mutant phage lambda integrase.

7. The method of claim 1, wherein the E. coli strain of (i) further comprises a nucleotide sequence encoding for single chain integration host factor 2 (scIHF2), preferably wherein scIHF2 has the amino acid sequence set forth in SEQ ID NO:9 or a functional variant or fragment thereof.

8. (canceled)

9. The method of claim 7, wherein IntC3 and scIHF2 are comprised in an expression cassette that is stably integrated into the genome of the E. coli strain and wherein, preferably, the expression of both, IntC3 and scIHF2, is stringently controlled by the same inducible expression control sequence.

10. The method of claim 9, wherein the expression cassette further comprises a selection marker, optionally flanked by recombination sites for later excision, and preferably wherein the expression cassette has the nucleotide sequence set forth in SEQ ID NO:1.

11. (canceled)

12. The method of claim 1, wherein the isolation in step (v) comprises digesting linearized and nicked DNA and extraction of the second circular DNA molecule.

13. The method of claim 1, wherein the DNA sequence of interest comprises one or more genes, preferably wherein at least one of the one or more genes is operably linked to expression control sequence(s).

14. (canceled)

15. The method of claim 1, wherein the second circular DNA construct comprising the DNA sequence of interest does not contain bacterial sequences, except the phage lambda integrase recombination sequence.

16. The method of claim 1, wherein the two directly repeated lambda integrase recombination sequences that are recombination substrates for the mutant phage lambda integrase are selected from the group consisting of attP and attB, attL and attB, attL and attL or functional variants thereof.

17. The method of claim 1, wherein the E. coli strain is E. coli strain MG1655 comprising a nucleotide sequence encoding a mutant phage lambda integrase (IntC3) having the amino acid sequence set forth in SEQ ID NO:2 or a functional variant or fragment thereof, wherein the expression of said nucleotide sequence is stringently controlled by an inducible expression control sequence.

18. An E. coli cell comprising a nucleotide sequence encoding a mutant phage lambda integrase (IntC3) having the amino acid sequence set forth in SEQ ID NO:2 or a functional variant or fragment thereof stably integrated into its genome, wherein the expression of said nucleotide sequence is stringently controlled by a genomic inducible expression control sequence.

19. The E. coli cell of claim 18, wherein the nucleotide sequence encoding a mutant phage lambda integrase (IntC3) is inserted into the genomic arabinose operon of E. coli.

20. The E. coli cell of claim 19, wherein integration is immediately downstream of the arabinose promoter by using the start codon of the endogenous araB gene as the start codon for the nucleotide sequence encoding a mutant phage lambda integrase.

21. The E. coli cell of claim 18, wherein the E. coli cell further comprises a nucleotide sequence encoding for single chain integration host factor 2 (scIHF2) stably integrated into its genome, preferably wherein scIHF2 has the amino acid sequence set forth in SEQ ID NO:9 or a functional variant or fragment thereof.

22. (canceled)

23. The E. coli cell of claim 21, wherein IntC3 and scIHF2 are comprised in an expression cassette that is stably integrated into the genome of the E. coli strain and wherein, preferably, the expression of both, IntC3 and scIHF2, is stringently controlled by the same inducible expression control sequence.

24. The E. coli cell of claim 18, wherein the E. coli cell is derived from E. coli strain MG1655.

25. The E. coli cell of claim 18, obtainable by stably integrating a nucleotide sequence encoding a mutant phage lambda integrase (IntC3) having the amino acid sequence set forth in SEQ ID NO:2 or a functional variant or fragment thereof into the genome of an E. coli cell, preferably an E. coli strain MG1655 cell, such the expression of said nucleotide sequence is stringently controlled by a genomic inducible expression control sequence.
